(19) United States
(12) Patent Application Publication
(10) Pub. No.: US 2024/0226523 A1
(43) Pub. Date: Jul. 11, 2024

(54) INFUSION PORT

(71) Applicant: CONNOR J. MACFARLANE, Medford, MA (US)

(72) Inventor: CONNOR J. MACFARLANE, Medford, MA (US)

(21) Appl. No.: 17/141,201

(22) Filed: Jan. 4, 2021

Related U.S. Application Data
(60) Provisional application No. 62/916,614, filed on Jan. 2, 2020.

(51) Int. Cl. A61M 39/02

(52) U.S. Cl. CPC A61M 39/02

(57) ABSTRACT

In accordance with the present invention, there is provided an infusion port comprising a base having a patient adhering engagement bottom surface, a top surface, and a through-bore whose outer perimeter comprises a cannula receiving groove. Mounted on the top surface of the base is a housing which encloses the through bore of said base. A housing needle is mounted within said housing, on the top surface of the base, and a cannula extends downward from the patient adhering engagement bottom surface, said cannula pressure fitted within the groove at the outer perimeter of the through-bore, such that the cannula, the through-bore, and the housing needle are co-linearly arranged to form a medicine delivery channel. The housing is further equipped with a lid and an o-ring, such that when the lid is closed it seals the housing needle and the medicine delivery channel within the housing. In use, the housing acts as a socket for receiving the medicine containing pen. Upon placement of the pen and pressing of the pen's diaphragm onto the housing needle within the housing, which pierces the diaphragm, and the pressing of the pen's plunger, medicine flows through the medicine channel, out the cannula and into the patient's body.

### CROSS-REFERENCE TO RELATED APPLICATIONS

This application claims priority from U.S. Provisional Application Serial No. 62/916,614 filed on Oct. 17, 2019, supplemented on Jan. 2, 2020, and incorporated by reference in its entirety, as if more fully set forth herein.

### BACKGROUND OF THE INVENTION

### Field of the Invention

The present invention is directed to the field of injection and infusion ports and methods for use thereof. More particularly, it relates to an infusion device specifically adapted for on-body mounting, and receiving medicine from a medicine pen, as for example, insulin from an insulin pen, and subcutaneously infusing it into an individual, without the use of a pen injection needle.

### Prior Art

Insulin is a hormone. Under normal conditions, the human body, i.e., beta cells in the pancreas, make(s) insulin to keep blood glucose levels within the normal range. Insulin enables cells to make energy by moving glucose from the bloodstream and into the body. If the body doesn't produce and have enough insulin, glucose builds up in the bloodstream instead of providing energy for the body.

With type 1 diabetes, the body does not make any insulin. Insulin has to be injected regularly every day, multiple times a day, to stay alive and healthy. With type 2 diabetes, the body does not make enough insulin, or the insulin that it makes does not work well. Thus, insulin injections may sometimes also be needed with type 2 diabetes to manage blood glucose levels.

The main devices for the injection of insulin into the body are syringes, insulin pens and insulin pumps. According to Endocrine News (April 2014), roughly 20% of insulin users in the U.S. wear an insulin pump, and 15% use insulin pens. In Europe the number of insulin pen users is much higher. About 66% to 75% use insulin pens. Patients prefer pens over vials and syringes for a myriad of reasons, including ease of use, less pain, and greater perceived social acceptance. Despite the fact that insulin pens are more expensive than vials and syringes.

There are several different types of insulin pens. Some insulin pens are disposable, with the insulin already in the pen. They have a plunger in them. Once the plunger has been pushed to the end of the pen, and the insulin has been used up, the pen must be discarded. Other insulin pens are non-disposable. They comprise a reusable pen which holds an insulin cartridge that can be inserted into the pen. To open and replace the insulin cartridge some of the non-disposable pens twist the two parts of the pen moving in the opposite direction. Other pens unscrew. The insulin cartridge is inserted into the pen with the smaller end going in first. Non-disposable pens also have a plunger. Once the plunger is at the end of the insulin cartridge, it must be replaced in the pen.

Every insulin pen, whether disposable, or non-disposable with a cartridge, comprises a pen needle. Pen needles are disposable needles that screw on to an insulin pen device. They allow insulin to flow from the cartridge and into the body. They are available in different lengths, ranging from 4-12.7 mm. However, research suggests that size 4-6 mm pen needles are best. The thickness of the needle (gauge) also varies—the higher the gauge, the finer the needle. It is important that a new pen needle is used with each injection. It is equally important to properly dispose of the pen needle after it has been used.

The advantage of using an insulin pen is that it requires fewer steps than a vial and syringe. All users need to do when using an insulin pen is to check the cartridge to ensure that there are no bubbles, prime the needle if there are, then dial up their dose and inject. In fact, most users are taught to prime every injection, because a needle does have dead volume, and so if they do not prime they will not get their full dose.

Insulin pens are more accurate than syringes, particularly for doses of 5 units of insulin or less. They may be larger than syringes, and they may produce a click that users can hear and feel as they dial a dose, giving them the confidence that they will be administering the correct dose. They may have a plunger that is larger and needles that are smaller gauge than needles used with syringes.

Administering insulin includes choosing the correct injection site. In adults, the best injection area is the abdomen because insulin injected into the abdomen area is injected right into subcutaneous tissue away from big muscles to become absorbed the same each time.

Individuals need both a needleless pen and a pen needle to perform an insulin injection. The needleless pen contains the pen cartridge with the insulin. The pen needle comprises a socket with a socket needle on one end to receive and pierce the diaphragm of the needleless pen when its tip is seated within the socket The individual pulls the cap off the pen to check the pen cartridge and determine if it contains enough insulin. Then they pull the tab off the pen needle and attach the pen needle to the pen by inserting the tip of the pen into the socket, piercing the diaphragm at the tip of the pen by pressing it into the needle within the socket and by twisting clockwise until it is tight. The other end of the pen needle opposite its socket end, has two caps, a bigger outer one and a narrower smaller inner one covering the needle per se. The needle caps must be removed to prime the insulin pen and then dial up the insulin dosage units. Upon insertion into the skin, the pen needle must remain on the skin straight in, perpendicularly to the skin; not at an angle. The insulin is injected by pushing the plunger button at the end of the pen all the way in, until the indicator dial goes to zero, and counting at least to ten (10) while leaving the pen in place. This will allow the insulin to flow into the subcutaneous region. Only after counting to ten should the needle and the pen be removed.

There are many emotional challenges with insulin injections including but not limited to fear, anxiety and stress as well as physical challenges such as bruising, scarring and pain. One of the biggest physical problems with both vials and syringes and pen needles is lipohypertrophy. Lipohypertrophy is damaged tissue that happens when insulin is injected into the same area many times. Scar tissue occurs under the skin with decreased blood flow to the area. Decreased blood flow means that insulin will not be absorbed, it will not work as well as it should. To prevent lipohypertrophy, it is very important not to inject in the same area over and over again. It is very important to keep moving the injection sites by rotating and injecting in different areas.

A number of devices have been developed to deal with the pain and discomfort of insulin injections and the reduction of lipohypertrophy. One such device is the Medtronic i-port. See https://www.medtronicdiabetes.com/products/i-port-advance. It is a small, discrete patch that sticks to the body like a bandage. It can be used for up to 3 days, making it unnecessary to prick the skin every time insulin is needed. It is used with vials, syringes and pens. It is the subject matter of at least one U.S. patent, i.e., U.S. Pat. No. 9,486,575 B2 titled INFUSION DEVICE (“the '575 Infusion Device”).

The '575 Infusion Device is specifically adapted for syringe and pen needle injections. In one embodiment, the infusion device comprises a body including an accessible surface having a single inlet port therein, an engagement surface having a single outlet port therein, a medication delivery channel extending between the single inlet port and the single outlet port, and an identification feature on the accessible surface of the body adjacent to the single inlet port. The single inlet port of the medication delivery channel is tapered thereby forming a funnel-shaped entry into the medication delivery channel. A cannula is coupled to the body at the single outlet port and is adapted for receiving medication from the single outlet port and transmitting the medication therethrough. Once the infusion device is mounted on the patient, the patient may use a syringe or a pen coupled to an injection needle for receiving an injection of one or more medications via the infusion device. The patient is spared having their skin pierced by the injection needle, because the patient now pierces the infusion device instead.

Another Medtronic on-body injector and a method for use thereof is disclosed in U.S. Pat. No. 9,545,477 (“the '477 On-Body Injector”). It includes a bolus reservoir, a bolus injection needle in fluid communication with the bolus reservoir, the bolus injection needle having a bolus injection needle tip aligned with the injection port, the bolus injection needle being slideably biased away from the injection port to define a gap between the bolus injection needle tip and the injection port, and a button operably connected to the bolus injection needle to slide the bolus injection needle along the injection axis. The button is operable to advance the bolus injection needle tip to close the gap and advance the bolus injection needle tip into the injection port. The button is further operable to advance a plunger through the bolus reservoir to deliver a predetermined bolus volume to the patient through the injection flow path.

Another device that has been developed to deal with the pain and discomfort of insulin injunctions and the reduction of lipohypertropy is an insulin pump. Insulin pumps are small, computerized devices that mimic the way the human pancreas works by delivering small doses of short acting insulin continuously (basal rate). The devices may also be used to deliver variable amounts of insulin when a meal is eaten (bolus). The basal insulin rates are usually set up in the pump, providing one or multiple basal settings programmed in the pump, based on the needs.

The pump, which is about the size of a smart phone or deck of cards, is worn on the outside of the body and delivers insulin through a tube (catheter), connected to a thin cannula, placed into the layer of fat under the skin, typically around the stomach area. The pump can be worn around the waist in a pump case, or attached to a belt or bra, in a pocket, or on an armband.

Hands-on training is needed to use an insulin pump. The training includes but is not limited to how to fill a pump reservoir, prime tubing, select an infusion site, change an infusion set, disconnect the device, calculate and program basal and bolus doses, troubleshoot potential problems, create backup plans in case of pump failure, and prevent diabetic ketoacidosis.

In general, there are two types of pump devices: a) traditional insulin pumps, which have an insulin reservoir (or container) and pumping mechanism, and attach to the body with tubing and an infusion set. The pump body contains buttons that allow the programming of insulin delivery for meals, specific types of basal rates, or suspend the insulin infusion, if necessary. And b) insulin patch pumps, which are worn directly on the body and have a reservoir, pumping mechanism, and infusion set inside a small case. Patch pumps are controlled wirelessly by a separate device that allows programming of insulin delivery for meals from the patch.

While the devices described above have reduced some of the pain and discomfort of insulin injections and the reduction of lipohypertrophy, they are still fraught with problems. For example, users of the Medtronic i-port, i.e., the '575 Infusion Device, experience a resistance to the insertion of the needle into the device and a back flow of insulin. Further, they still have to properly dispose the pen or syringe needles after use. Users of the '477 On-Body Injector may experience mechanical failure of the button and the plunger within the device, which may in turn contribute in the device's failure to deliver any insulin at all.

Similarly, users of insulin pumps simply replace one set of problems with another. They turn over their own precise measurement of insulin, and the security of manual delivery associated with direct injections with a syringe to the insulin pump. Nevertheless, they worry about receiving an incorrect dosage of insulin due to pump malfunctioning, degradation of the insulin within the pump reservoir (e.g., due to heat), bubbles in the reservoir/supply tube of the pump (e.g., due to agitation) and inherent limitations of an electro-mechanical device.

As the pump is a continuous delivery device, the users may not know that they are receiving an incorrect dosage of insulin until a lengthy period of time has passed, resulting in dangerous blood-sugar levels. Their self-confidence and self-esteem is seriously compromised because the insulin pump, which is about the size of a typical pager, must be worn essentially 24 hours per day. Finding an inconspicuous yet convenient place to wear the pump can be difficult, particularly during summer, during athletic events, or even during intimate circumstances.

Finally, users of insulin pumps become financially challenged as a result of the cost of the insulin pump—about $8000 for the pump, plus disposable supplies. Though insurance plans generally cover insulin pumps, the considerable price of the pump adversely affects insurance premiums. In addition, users may still have the responsibility of paying for at least a portion of the pump, even if they have health insurance.

Accordingly, there still exists a great need to address both the emotional and physical challenges experienced with insulin injections, namely the fear, anxiety, stress, bruising, scarring and pain. There still exists a need to reduce lipohypertrophy caused by multiple insulin injections into the same area many times to insure that insulin will be absorbed. There still exists a need to address the decrease of self-confidence and self-esteem caused by the use of insulin pumps. There is a serious need to reduce the treatment costs usually associated with insulin pumps. Finally, there still exists a need to deliver insulin without the use of syringe needles or insulin pen needles, and to protect the environment by not polluting it with pen needles or syringe needles, after their use for an injection.

### SUMMARY OF INVENTION

Accordingly, it is an object of the present invention to provide a device that addresses both the emotional and physical challenges experienced by patients who use syringes and needles or injection pens to self-medicate, particularly patients who self-inject insulin, namely the fear, anxiety, stress, bruising, scarring and pain that occur from multiple injections.

It is another object of the present invention to provide a device that reduces lipohypertrophy caused by multiple injections in the same area, particularly by multiple insulin injections to insure that insulin will be absorbed.

It is yet another object of the present invention to provide a device that addresses the decrease of self-confidence and self-esteem caused by the use of insulin pumps.

It is still another object of the present invention to provide a device that reduces the treatment costs usually associated with insulin pumps.

It is a further object of the present invention to provide a device that delivers insulin without the use of syringe needles or insulin pen needles, and protects the environment by not polluting it with pen needles or syringe needles, after their use for an injection.

It is yet a further object of the present invention to provide a device that remains in place for an extended period of time thereby permitting the patient to deal with only one injection type needle over 3 or more days, rather than multiple times a day.

In accordance with the present invention, there is provided an infusion port comprising a base having a patient adhering engagement bottom surface, a top surface and a through bore whose outer perimeter comprises a cannula receiving groove. Mounted on the top surface of the base is a housing which encloses the through bore of said base. A housing needle is mounted within said housing, on the top surface of the base, and a cannula extends downward from the patient adhering engagement bottom surface, said cannula pressure fitted within the deep groove at the outer perimeter of the through bore, such that the cannula, the through-bore and the housing needle are co-linearly arranged to form a medicine delivery channel. The housing is further equipped with a lid and an o-ring, such that when the lid is closed it seals the housing needle and the medicine delivery channel within the housing. In use, the housing acts as a socket for receiving the medicine containing pen. Upon placement of the pen and pressing of the pen's diaphragm onto the housing needle within the housing, which pierces the diaphragm, and the pressing of the pen's plunger, medicine flows through the medicine channel and out the cannula into the patient's body.

These and other objects, advantages, features, and characteristics of the invention will be apparent from the following description of the preferred embodiments, considered along with the accompanying drawings.

### BRIEF DESCRIPTION OF THE DRAWINGS

It is believed that the present invention will be better understood from the following detailed description taken in conjunction with the accompanying drawings, in which the numerals represent identical elements and wherein:

FIG. 1 is a side plan view of the deployed inventive Infusion Port in actual use, in the open position.

FIG. 2 is a front plan view of the deployed inventive Infusion Port of FIG. 1.

FIG. 3 is a top, three-dimensional view of the deployed inventive Infusion Port of FIG. 1.

FIG. 4 is a bottom plan view of the deployed inventive Infusion Port of FIG. 1.

FIG. 5 is a side plan view of the medicine delivery channel within the inventive Infusion Port.

FIG. 6 is a bottom plan view of the inserter shell.

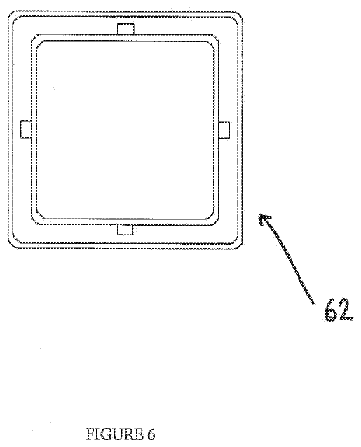

FIG. 7 is a top view of the plunger/stabilizer combination for the inserter needle used to deploy the cannula within a subcutaneous region of a patient.

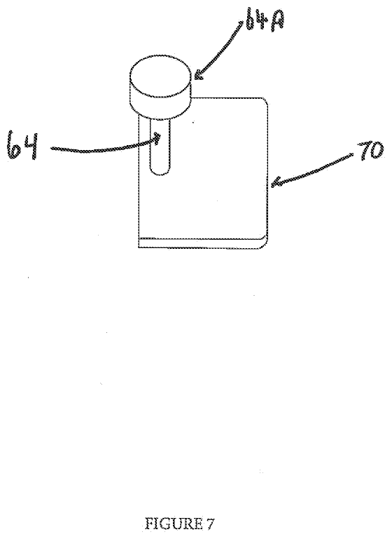

FIG. 8 is a top plan view of the inserter cover.

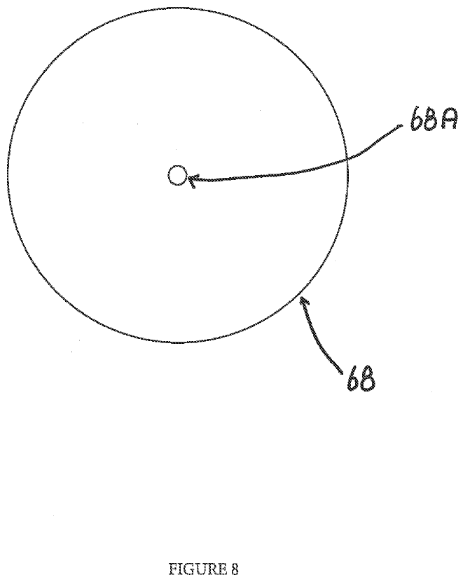

FIG. 9 is a side plan view of the inserter cover.

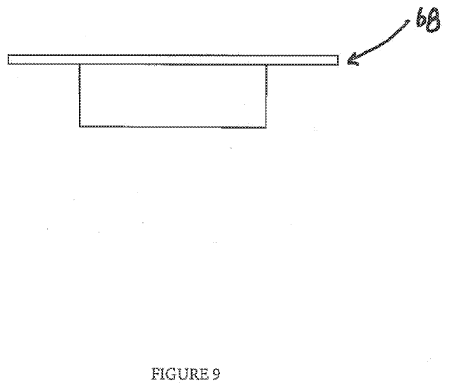

FIG. 10 is a bottom three-dimensional view of the inserter cover.

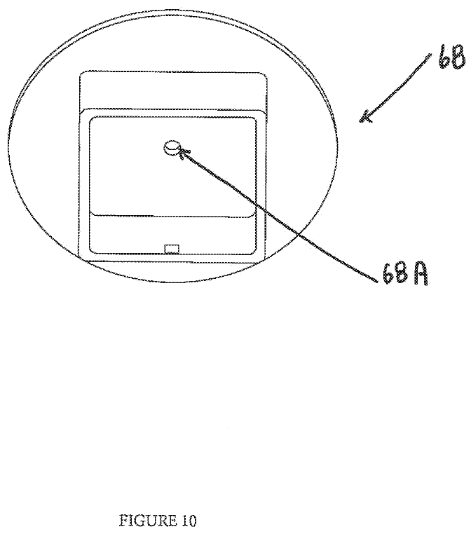

FIG. 11 is a side plan view of the plunger stabilizer of FIG. 7.

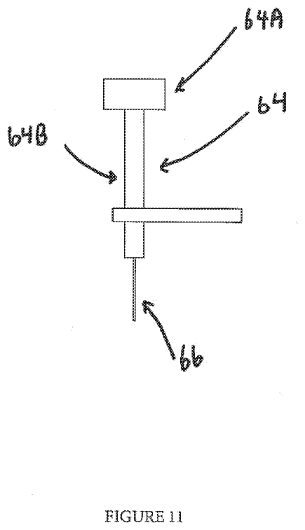

FIG. 12 is a side view of the through bore acting as a locking mechanism on the base of the Inventive Fusion Port.

FIG. 13 is a plan view of a second embodiment of the medicine delivery channel within the inventive Infusion Port.

FIG. 14 is a three dimensional perspective of the inventive Infusion Port within its Insertion/Deployment Apparatus (collectively the “Deployment Assembly”).

FIG. 15 is a front plan view of the Deployment Assembly of FIG. 14.

FIG. 16 is a side plan view of the Deployment Assembly of FIG. 14.

FIG. 17 is a top plan view of the Deployment Assembly of FIG. 14.

FIG. 18 is a bottom plan view of the Deployment Assembly of FIG. 14.

FIG. 19 is a cross-section, cut-away plan view of the Deployment Assembly taken along line A-A′ of FIG. 14, showing the internal structure of the inventive Infusion Port together with its deployment device.

FIG. 20 is an exploded view of the Insertion/Deployment Assembly of FIG. 14, showing the inventive Infusion Port together with the components of the deployment device.

FIG. 21 is a three dimensional perspective view of the deployment device plunger of FIG. 20.

FIG. 22 is a front plan view of the deployment device plunger of FIG. 20.

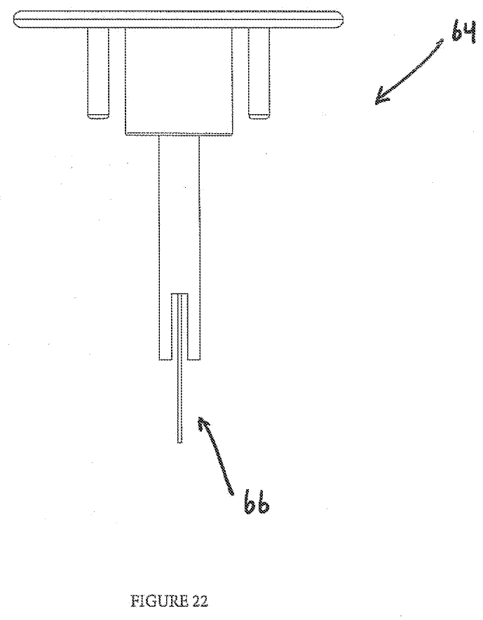

FIG. 23 is a side plan view of the deployment device plunger of FIG. 20.

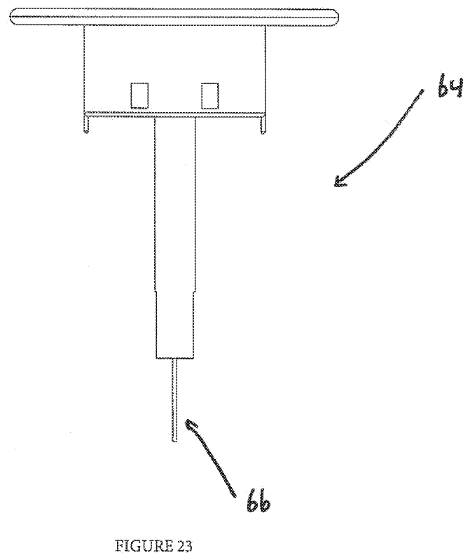

FIG. 24 is a top plan view of the deployment device plunger of FIG. 21.

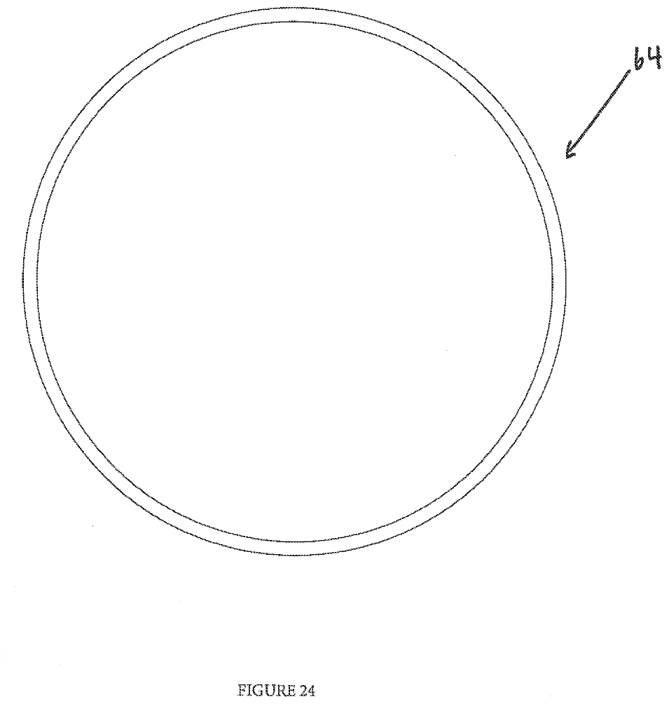

FIG. 25 is a bottom plan view of the deployment device plunger of FIG. 21.

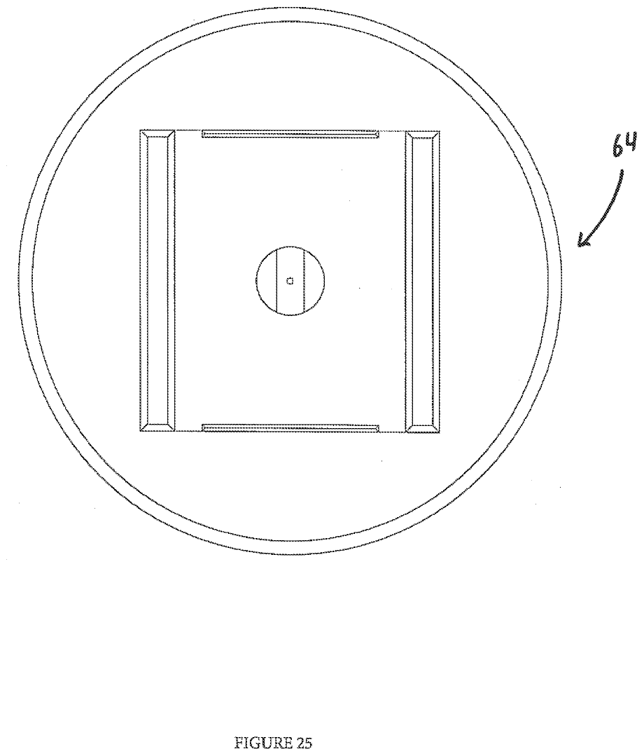

FIG. 26 is a cross-section, cut-away plan view of the deployment device plunger of FIG. 21 taken along line B-B′.

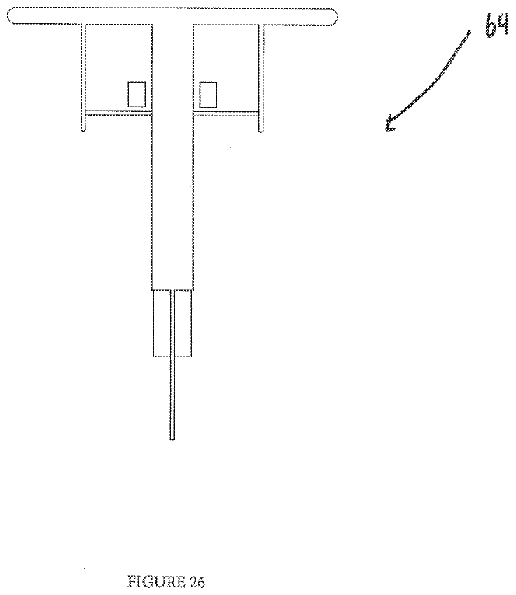

FIG. 27 is a three dimensional perspective view of the deployment device housing of FIG. 20.

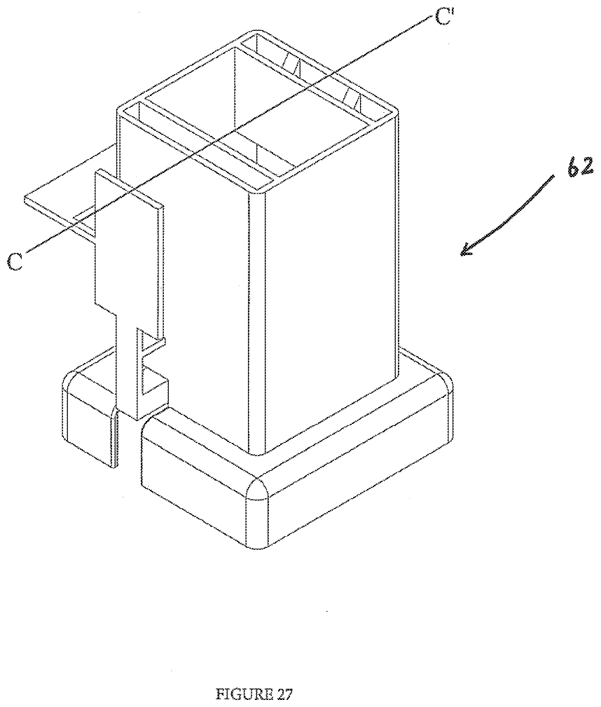

FIG. 28 is a front plan view of the deployment device housing of FIG. 27.

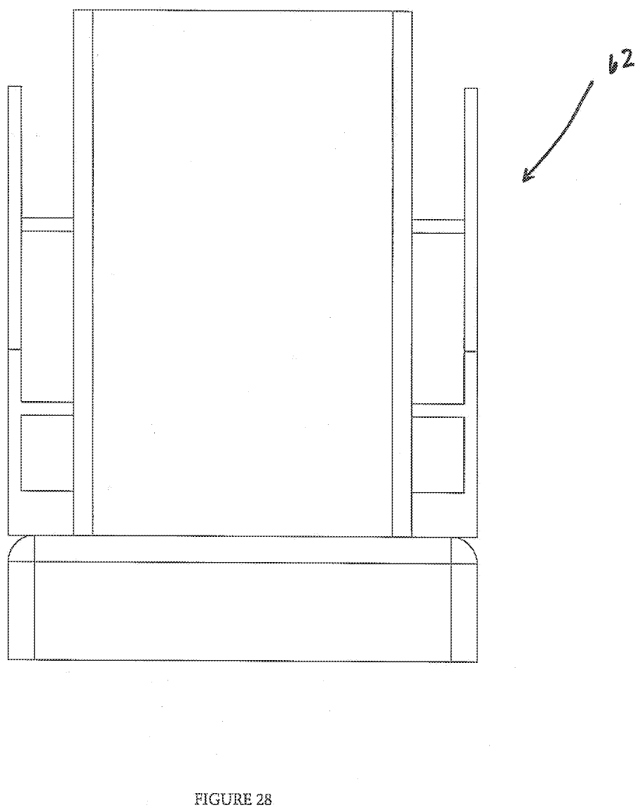

FIG. 29 is a side plan view of the deployment device housing of FIG. 27.

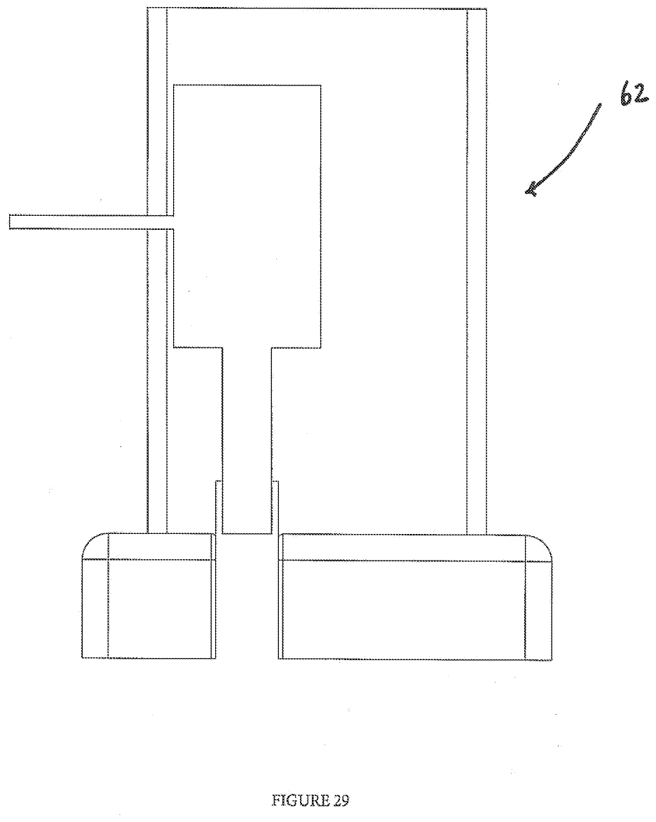

FIG. 30 is a top plan view of the deployment device housing of FIG. 27.

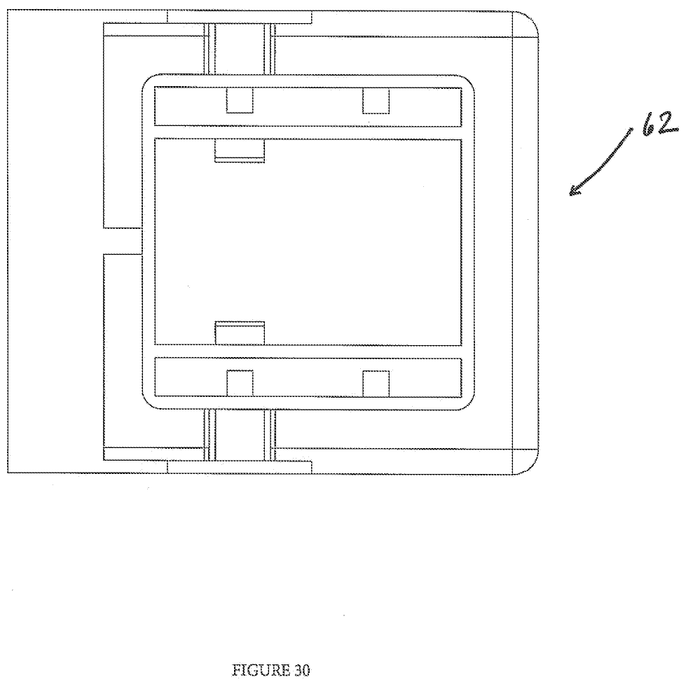

FIG. 31 is a bottom plan view of the deployment device housing of FIG. 27.

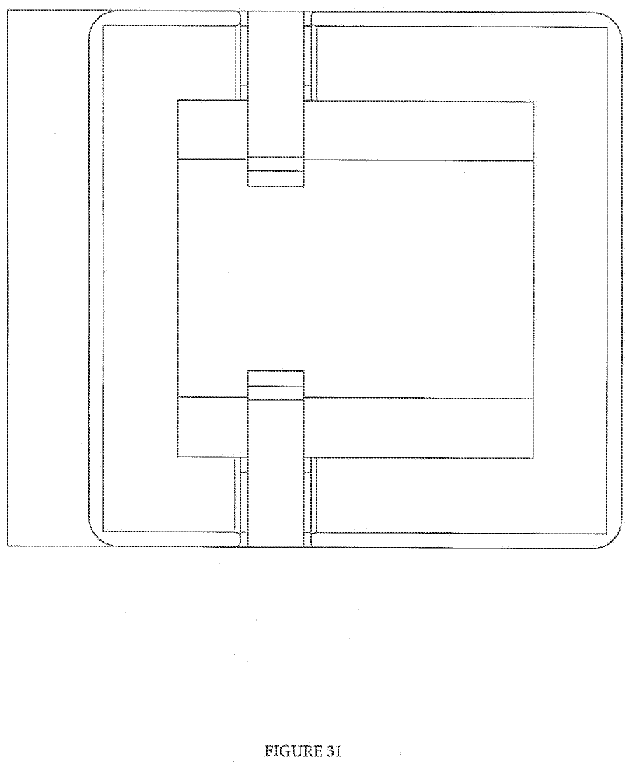

FIG. 32 is a cross-section, cut-away plan view of the deployment device housing of FIG. 27 taken along line C-C′.

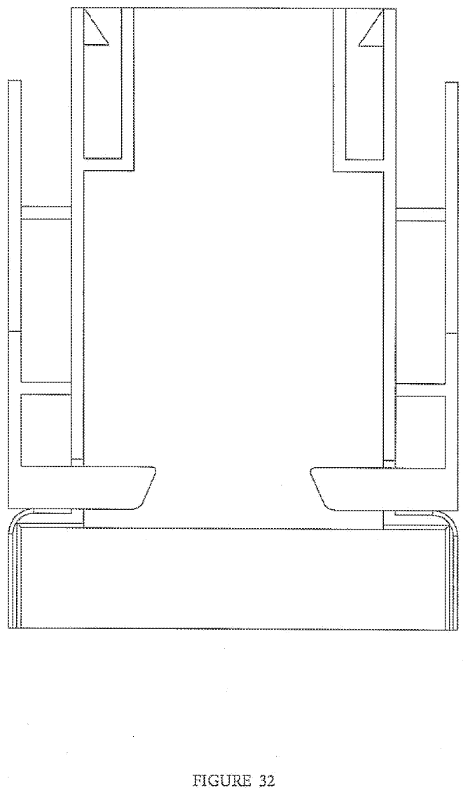

FIG. 33 is an angled three dimensional perspective view of the inventive infusion port of FIG. 20.

FIG. 34 is an angled three dimensional perspective view from the top of the inventive infusion port of FIG. 20.

FIG. 35 is a front plan view of the Infusion Port of FIGS. 33 and 34.

FIG. 36 is a side angled plan view of the Infusion Port of FIGS. 33-34.

FIG. 37 is a side plan view of the Infusion Port of FIGS. 33-34.

FIG. 38 is a top plan view of the Infusion Port of FIGS. 33-34.

FIG. 39 is a bottom plan view of the Infusion Port of FIGS. 33-34.

FIG. 40 is a back plan view of the Infusion Port of FIGS. 33-34.

FIG. 41 is a cross-section, cut-away plan view of the Infusion Port of FIGS. 33-34 taken along line D-D′.

FIG. 42 is a close-up plan view of the medicine delivery channel of the inventive Infusion Port within the area denoted by the letter Z in FIG. 41.

LIST OF ELEMENTS AND THEIR RESPECTIVE
IDENTIFYING NUMERALS

| NUMBER | ELEMENT |
|---|---|
| 10 | Infusion Port (the invention) |
| 20 | Base |
| 20A | top surface of the Base |
| 20B | Bottom surface of the Base |
| 20C | Removable Protective Shield (not shown in figures) |
| 26 | through-bore |
| 27 | through-bore, cannula receiving groove |
| 28 | through-bore bushing |
| 30 | Housing |
| 32 | Housing outer straight edge wall defining the back of the |
| | Housing |
| 34 | Circular inner cavity within Housing |
| 36 | Two female indentations |
| 40 | lid |
| 40A | top surface of lid |
| 40B | bottom surface of lid |
| 42 | lid-housing pivot joint |
| 44 | O-ring |
| 46 | Lip of the lid |
| 48 | drop down ridge |
| 50 | Medication Delivery Apparatus, where e.g., |
| | the medication can be Insulin, monoclonal |
| | antibodies, or immune system |
| | modulators. |
| 52 | Soft Cannula |
| 54 | Means to connect Soft Cannula to Hard Needle (“connecting |
| | means”) |
| 56 | Hard Needle |
| 57 | Flat disk shaped top |
| 58 | Cone shaped, funnel shaped, arrow shaped bottom |
| 60 | Insertion/Deployment Apparatus |
| 62 | Inserter Shell |
| 62 A | male bosses or protrusions or nubs |
| 64 | plunger |
| 64A | plunger head |
| 64B | plunger shaft |
| 66 | Insertion needle |
| 68 | Inserter cover |
| 68A | plunger apperture on Inserter cover |
| 70 | safety tab |
| 72 | upper wing |
| 74 | lower wing |
| 76 | male nubs/protrusions |

### DETAILED DESCRIPTION OF THE INVENTION

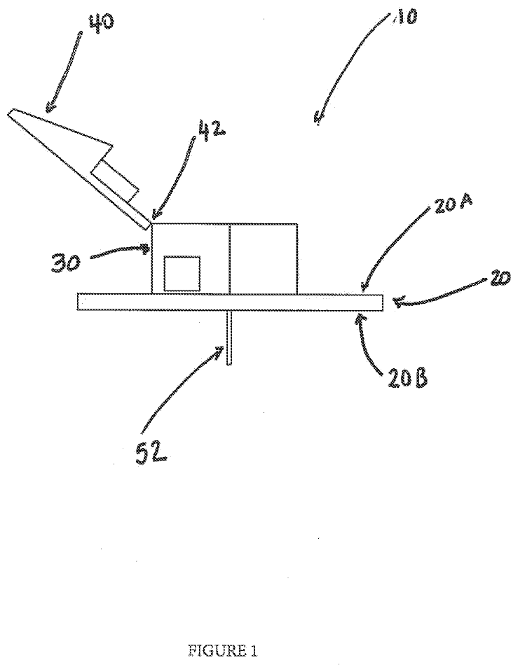

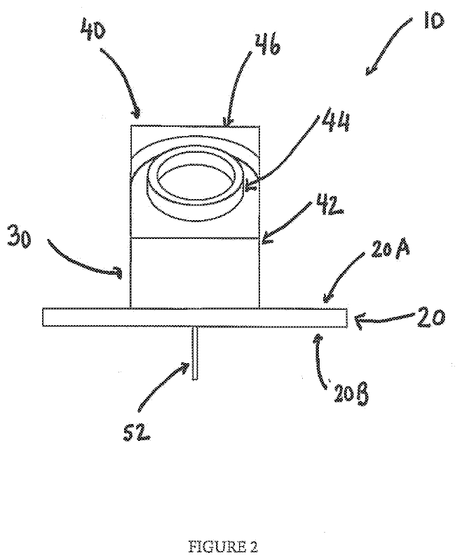

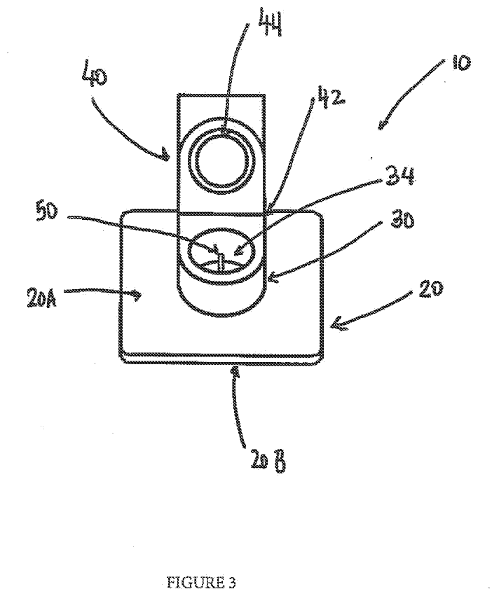

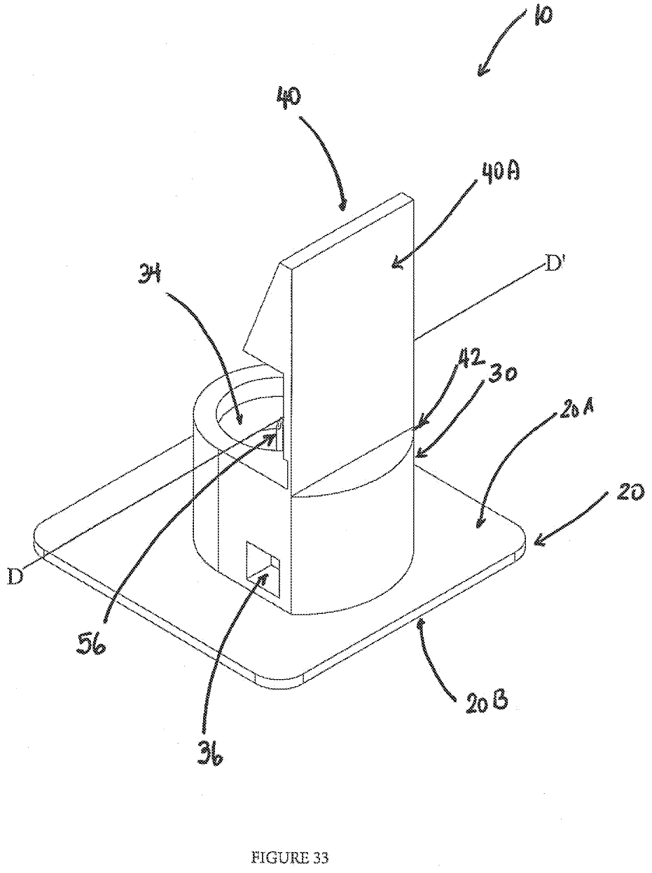

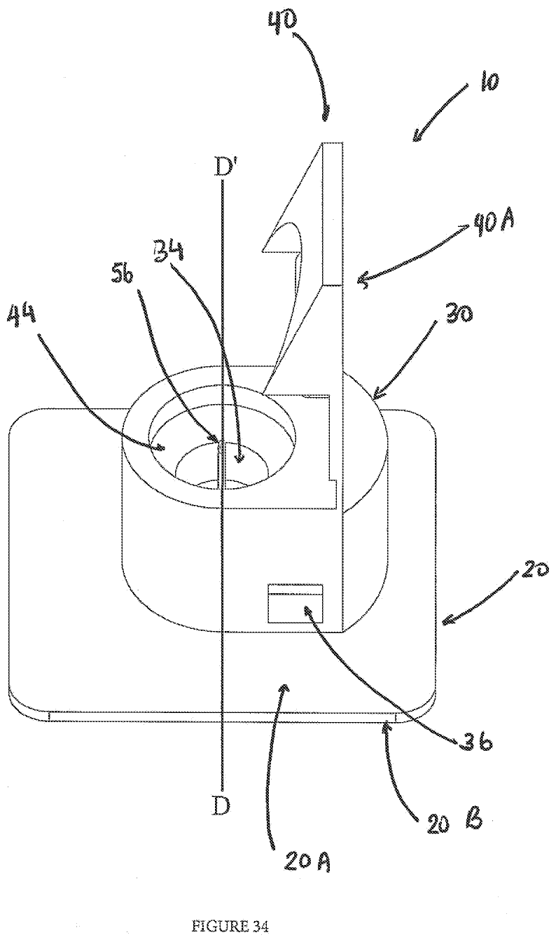

Referring more specifically to FIGS. 1-3 and FIGS. 33-34, they generally depict at 10 at least one of the embodiments of the inventive Infusion Port for the subcutaneous delivery of medicine into a patient's bloodstream. In the preferred embodiment the patient is a diabetic and the inventive Infusion Port 10 is specifically adapted for receiving insulin from an insulin pen and delivering the insulin to the patient, but without the use of an insulin pen needle.

As discussed in greater detail below, such infusion port is beneficial to any patient that requires receiving any medication via a medication pen, as for example insulin via an insulin pen on a daily basis, at least once daily. Other examples of medications that may be delivered via a medication pen are immune system modulators every other day, or monoclonal anti-bodies.

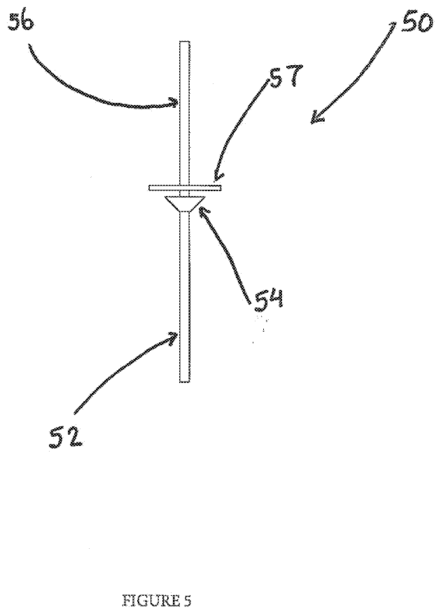

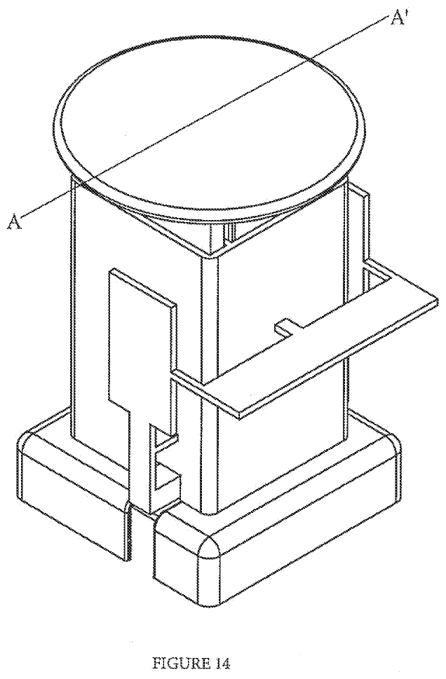

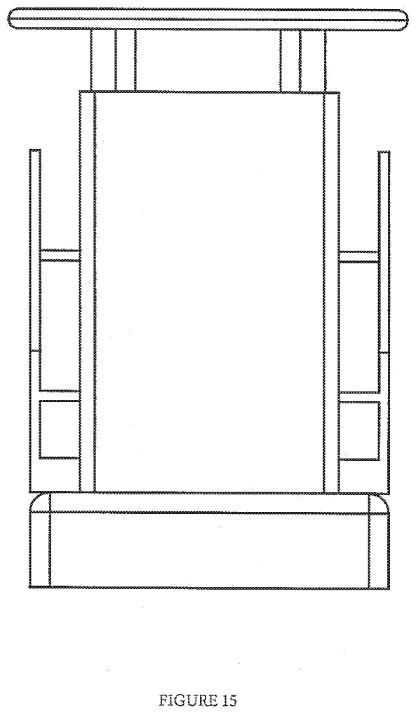

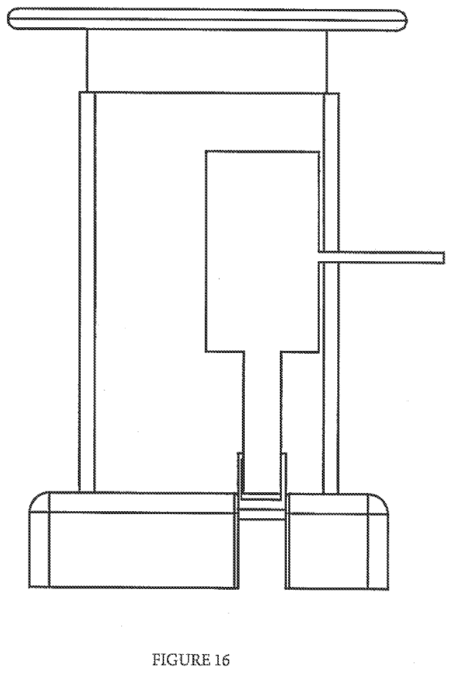

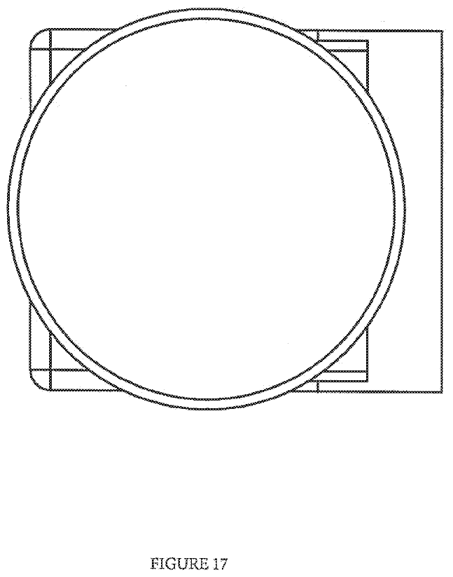

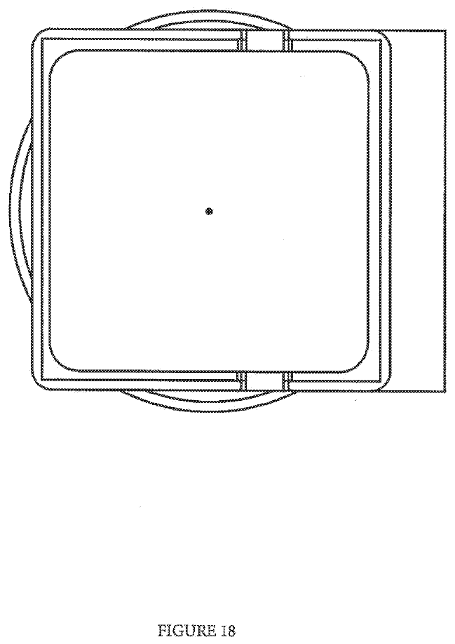

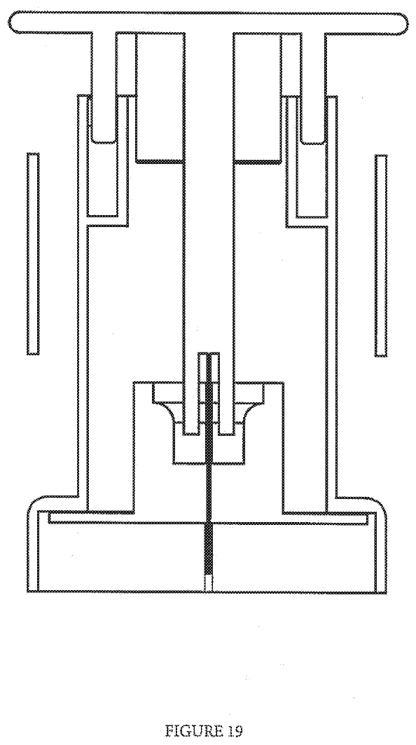

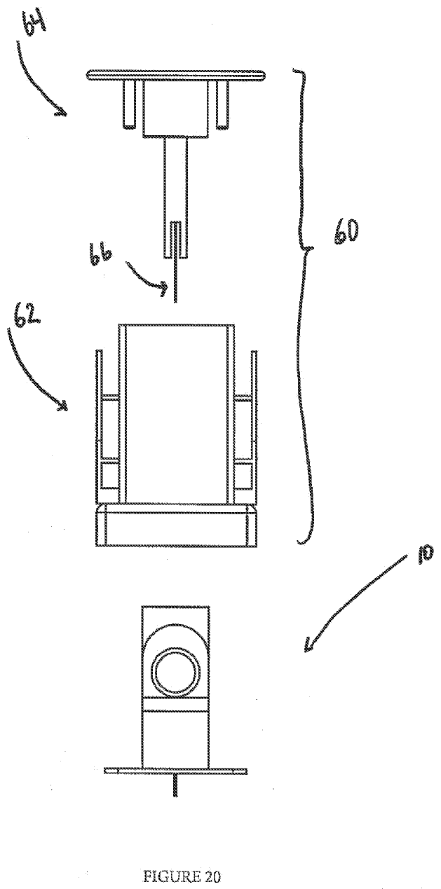

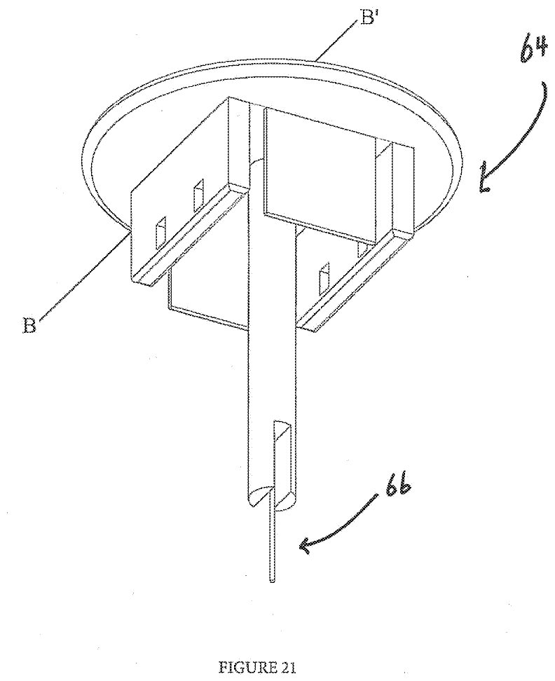

The inventive infusion port 10, having a medication delivery apparatus 50, see FIGS. 5, 13, 41 and 42, as for example an insulin delivery apparatus 50, is adhesively fixed on the skin of a patient and deployed in a manner that allows the medication delivery apparatus 50 to extend into or through the subcutaneous tissue of the patient. Once it is adhesively mounted on the patient, they may use a medication pen to access the medication delivery apparatus 50 for the delivery of medication, but without using the medication pen's corresponding medication pen injection needle. The patient is spared having their skin pierced by the pen injection needle. After an initial skin piercing by an insertion needle 66 of the deployment assembly, see FIGS. 14-21 all subcutaneous deliveries of medication to the patient are facilitated by the medicine pen being engaged with the infusion port, but without a medicine pen needle, instead of through the skin of the patient. The patient will only be subjected to piecing of the skin by a needle, every few days instead of multiple times a day, when replacing an existing mounted infusion port with a new infusion port.

An infusion port in accordance with the present invention permits patients who might not otherwise choose direct injection with an insulin pen, as their primary mode of medication delivery, due to any number of reasons (e.g., an aversion to needles, an intolerance to bruises at an injection site, etc.), to now do so. Through the use of such an infusion port, a patient may enjoy the precise measurement of a medication and/or the security of manual delivery afforded by direct injection with a syringe. It should be noted that direct injection is one of the most reliable methods of self-delivery that a patient can choose. This reliability is due at least in part to this precise measurement of the medication and the security of manual delivery. In essence, a patient has direct control over when, where and how much medication they are receiving. Accordingly, an infusion port in accordance with the present invention enables patients to properly medicate themselves, thus maintaining their health and mental well being.

Another advantage of an infusion port in accordance with the present invention is the ability to easily conceal the infusion port, without hindering access to it. The size and profile of the infusion port permits it to be worn inconspicuously under clothing at various injection points on the patient's body. While not directly related to a patient's physiological health, being able to readily conceal the infusion port under nearly any garment goes a long way to enhancing the mental well-being of human patients.

Still another advantage of an infusion port in accordance with the present invention, is its cost. Its cost is a fraction of other delivery devices intended to reduce anxiety and/or discomfort associated with direct injections with a syringe. Its relatively low cost will be of benefit to patients, doctors and insurance companies.

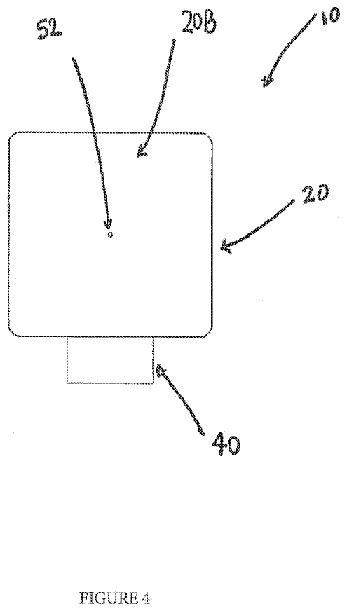

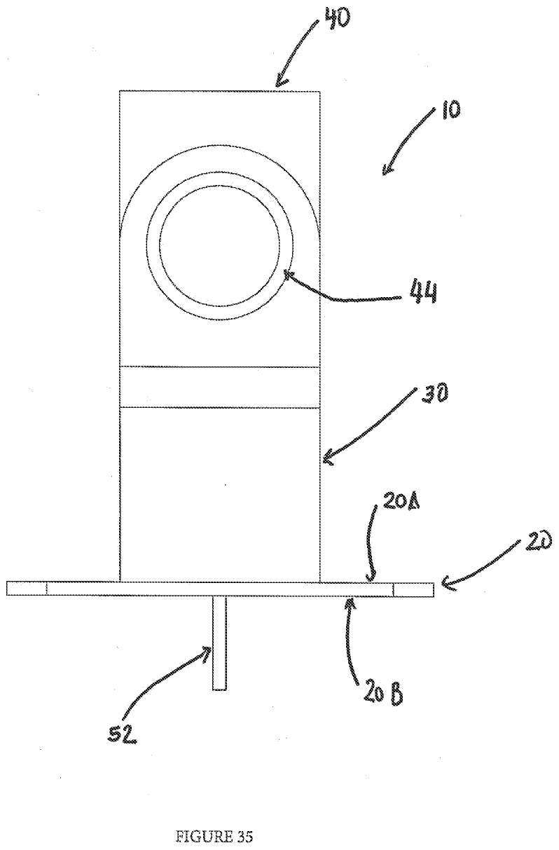

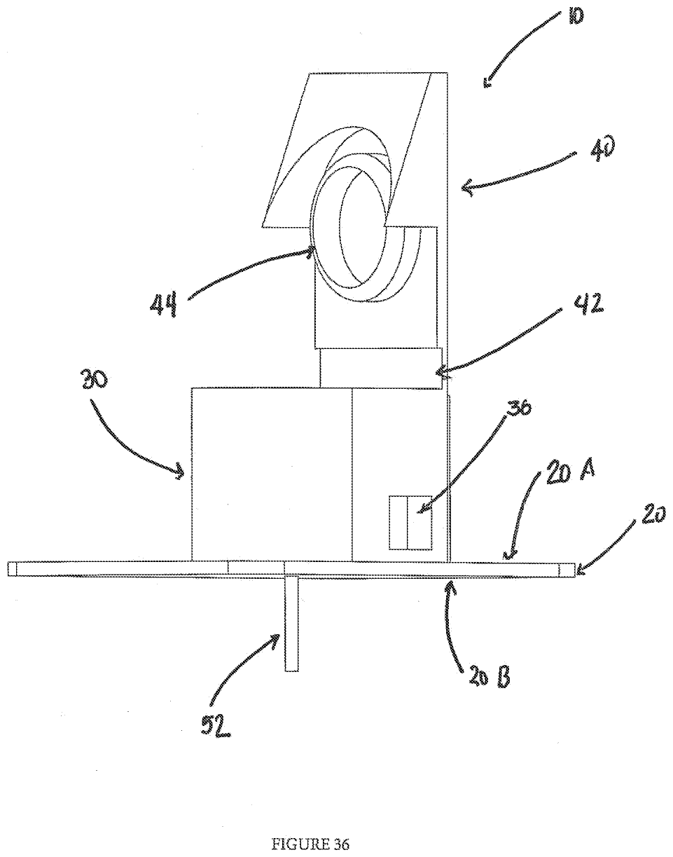

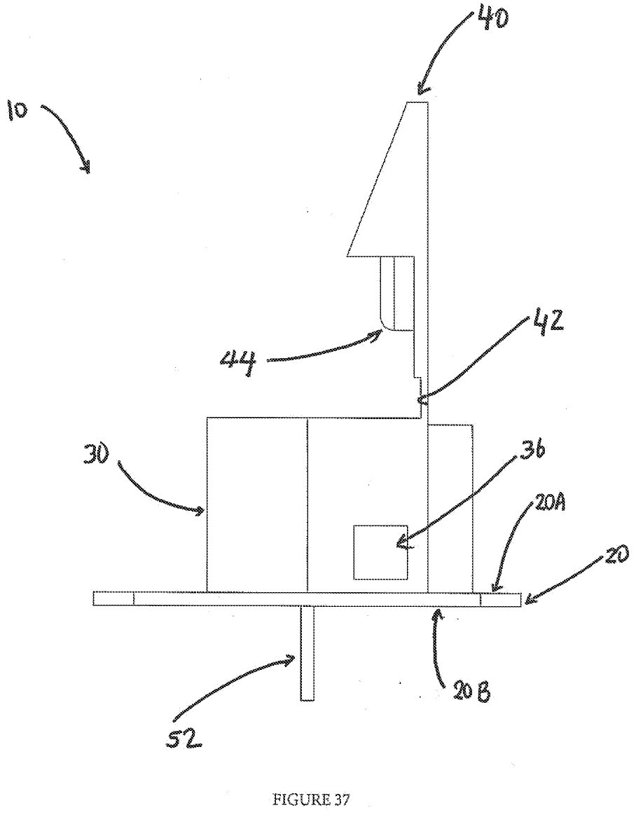

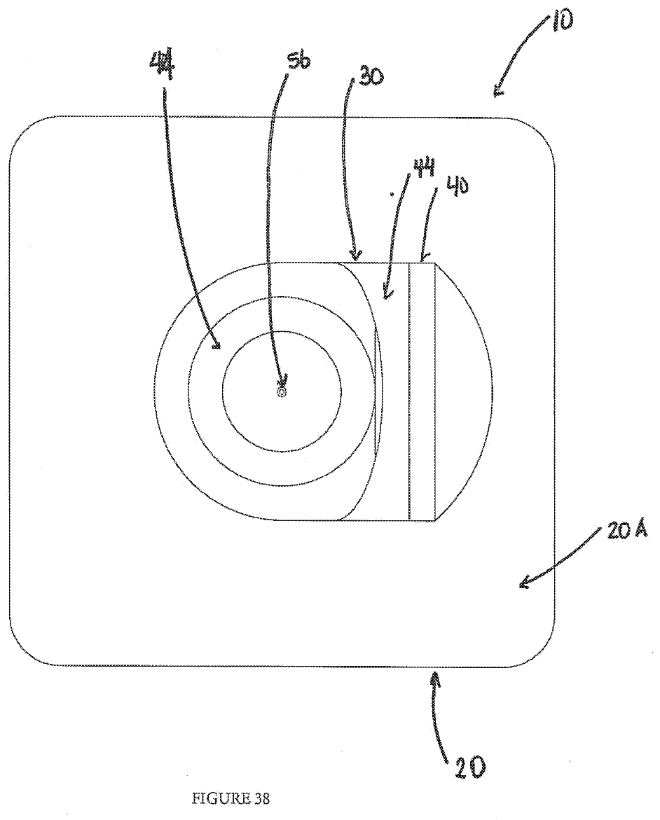

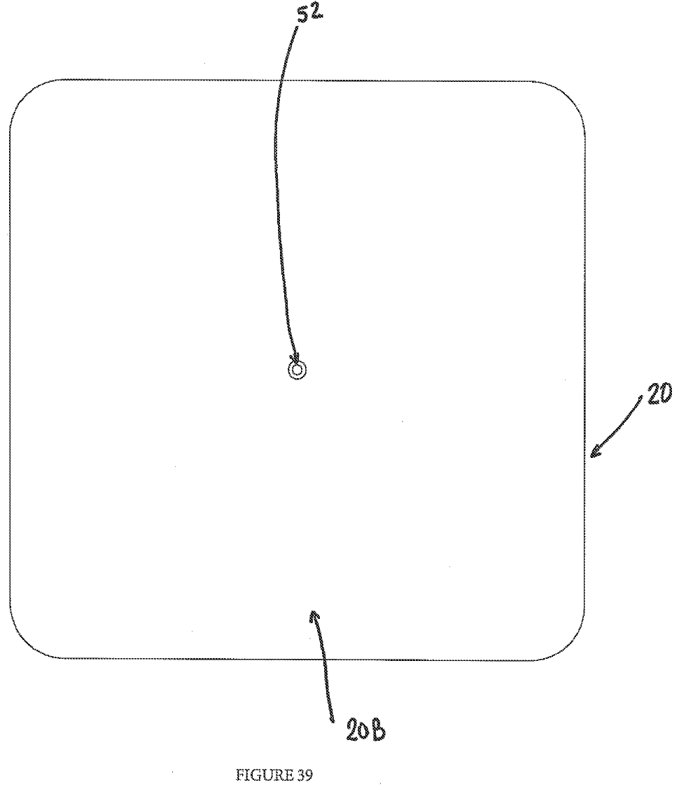

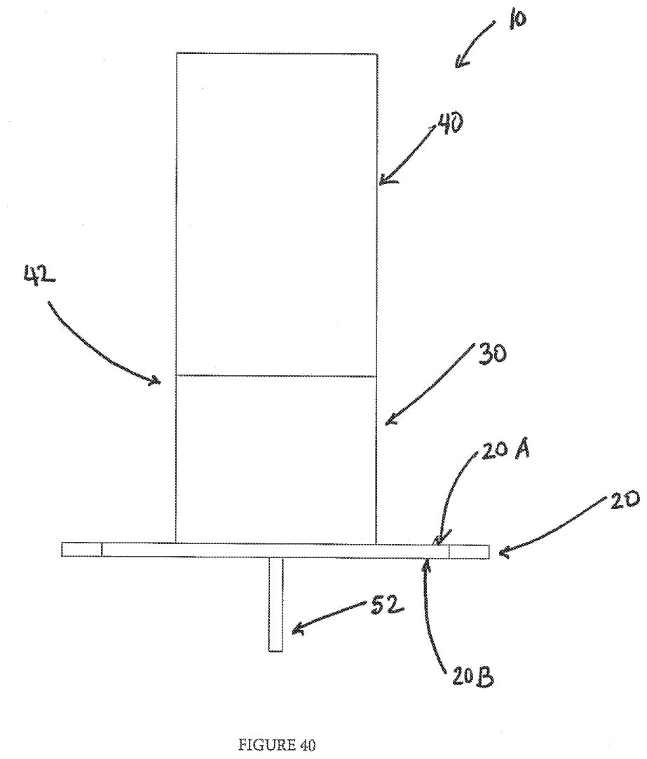

Referring more specifically to FIGS. 1-4 and FIGS. 33-40, the Infusion Port 10 in accordance with the present invention comprises a base 20 having a top surface 20A and a bottom surface 20B. It further comprises a housing 30 having a top end 30A and a bottom end 30B mounted at the approximate center of said top surface 20A of said base 20, such that said base 20 forms a flange around the entire perimeter of said housing 30. A lid 40 is hingedly connected via for example a live hinge 42 to said top end 30A of said housing 30. A Medication Delivery Apparatus 50 is disposed within said base 20 and releasably sealed within said housing 30 by said lid 40.

Said bottom surface 20B of said base 20 is provided with an adhesive protected by a removable protective shield liner 20C (not shown), which upon its removal provides said base 20 an adhering engagement bottom surface 20B capable of being securely but removably fixed to the skin surface of a patient during mounting and deployment of the infusion port 10. The adhering engagement bottom surface 20B secures the infusion port 10 to the patient's body. Although base 20 is depicted as being essentially planar, it may have a profile other than essentially planar. Further in one preferred embodiment, it is flexible and bendable so as to be capable of being mounted on any location on the patient's body suitable for the receiving of medication from a medication pen. The overall dimensions of said base 20 can be increased or decreased for improved, augmented adhesion to a patient's skin, by increasing or decreasing the dimensions of the flange beyond the outer perimeter of said housing 30. In one embodiment said base 20 may comprise a soft, bendable plastic. In another embodiment said base 20 may be a textile.

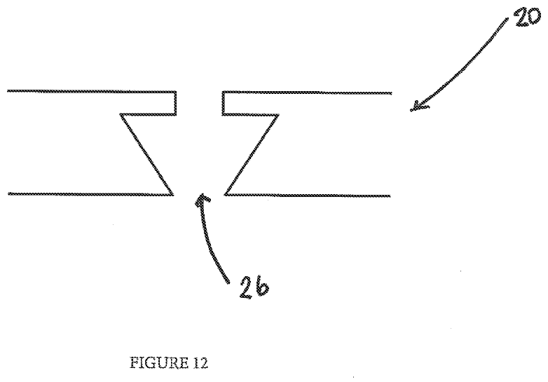

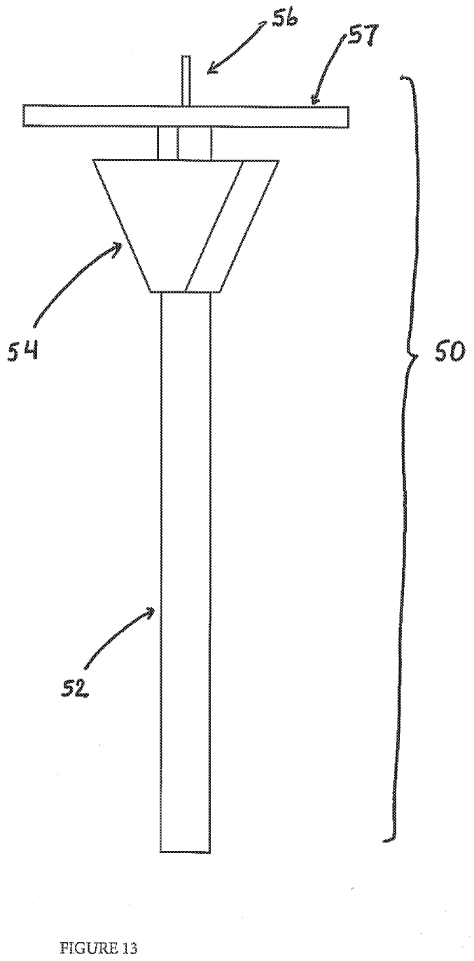

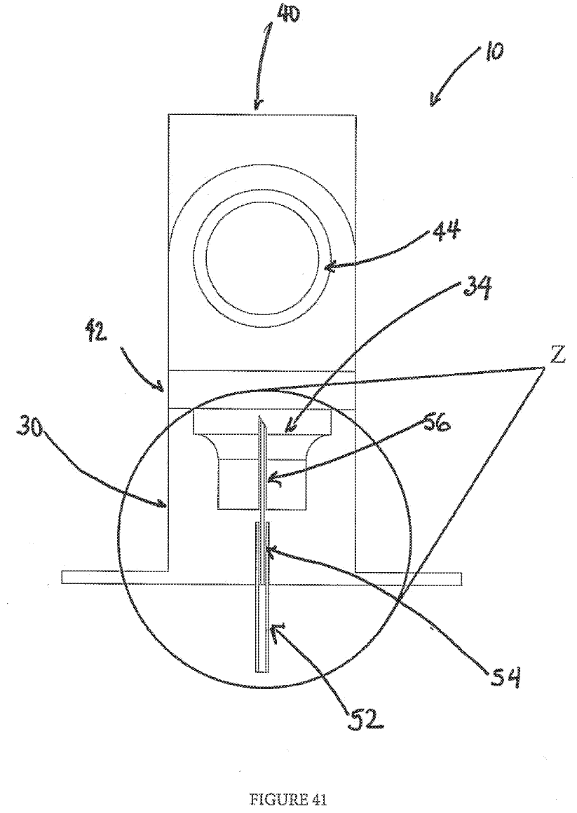

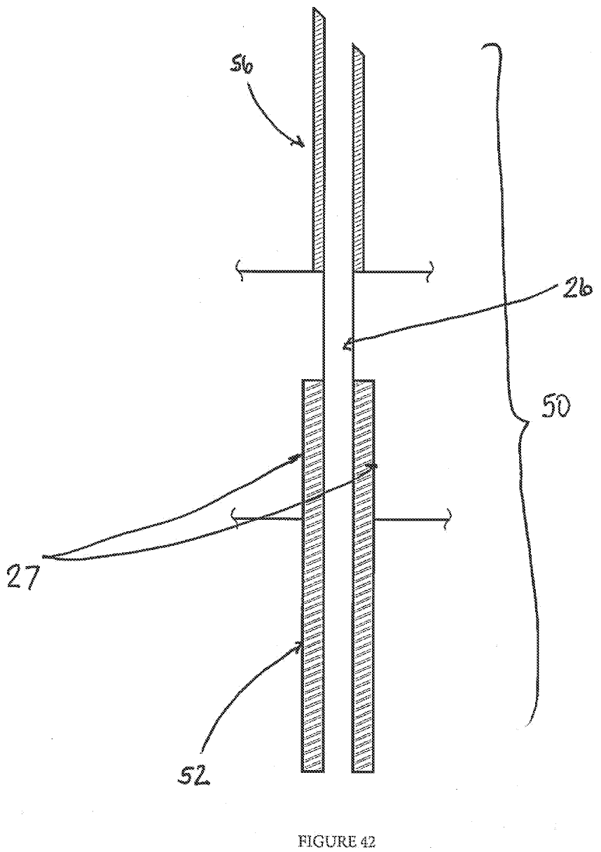

Said base 20 is further provided with a through-bore 26 which begins at the bottom surface 20B on said base 20, vertically traverses the height of base 20 to end at the top surface 20A of said base 20 within and enclosed by said housing 30. In a first embodiment of the Infusion Port, the through-bore 26 may be funnel-shaped and tapered in such a manner that just like a funnel, it is wider at its top end near the top surface 20A and narrower at its bottom end, see FIG. 12. Optionally, extending inwardly, towards the center of the tapered, funnel-shaped through-bore 26 and around the entire perimeter of its wider upper end, there may be a flange, also known as an inwardly projecting flat rim or collar or rib designed to lock in place the medication delivery apparatus 50 depicted in FIGS. 5 and 13. In a second embodiment of the Infusion Port as depicted in FIGS. 41-42, the outer perimeter of said lower end of said through-bore 26 is provided with a groove 27 and a bushing 28.

Optionally, in any of the preferred embodiments of said infusion port 10, said housing 30, mounted on and having walls extending above said base 20 and surrounding said through-bore 26, may be shaped like a “basketball key” having one outer straight edge wall 32 defining the back of said housing 30, perpendicular to an outer u-shaped edge wall, and having inner walls that define a totally circular inner cavity 34 having a top open end and a bottom closed end. Two female indentations 36 are provided, one on each side of said outer u-shaped edge wall, towards the back of said housing adjacent and parallel to said outer straight edge wall 32, immediately adjacent and perpendicular to said base 20. Optionally, said inner cavity or chamber 34 may be further provided with an O-ring 44, which may function to seal the chamber and act as a stabilizer of the medication pen, once it is inserted into the housing.

As is set forth above, the lid 40 is hingedly connected to said top end 30A, along said outer straight edge wall 32 defining the back of said housing 30. It comprises a hard rubber board-like, planar member having a top surface 40A and a bottom surface 40B, said planar member capable of being moved from an open position to a closed position vis-à-vis said housing 30. Mounted on the bottom surface 40B is a second O-ring 44, whose diameter corresponds to the inner diameter of the cavity 34 of said housing 30. Said O-Ring 44 abuts, presses against the wall of and seals said cavity 34 to form an inner closed chamber within said housing, when said lid 40 is in the closed position. Said lid 40 is further provided with a lip 46, which is used to push said lid open, and a drop down ridge 48 forming the inner partially curved wall of said lip, permitting the lip 46 of the lid to encircle the front curved wall of said housing and frictionally keep the lid closed, when said planar member of lid 40 is moved to the closed position.

Said medication delivery apparatus 50 comprises a soft cannula 52, a hard needle 56 and means 54 for connecting said soft cannula 52 to said hard needle 56. Each of said soft cannula 52, said hard needle 56 and said means 54 for connecting both, have a channel, respectively. Upon contiguous connection or assembly, all three channels become collinear to form one single channel along one essentially straight, common longitudinal axis. The channel acts as a conduit for receiving and transferring medication from a medication pen through the soft cannula 52 and into the blood stream.

In a first embodiment of the inventive Infusion Port 10, where said through-bore 26 is funnel-shaped, see FIG. 12, the medication delivery apparatus 50 of FIGS. 5 and 13 is assembled separately from said base 20. Said means 54 for connecting said soft cannula 52 to said hard needle 56 comprises a small tubular sleeve-like component of compressible material that has a flat disk-shaped top 57 connected to a cone shaped lower half 58. Said hard needle 56 extends upwardly from said flat disk-shaped top 57. Said soft cannula 52 extends downwardly from said cone shaped lower half 58. Upon its full deployment on a patient, the soft cannula 52 remains inserted within the patient's body extending below the through-bore 26 of said base 20 and said hard needle 56 remains above said flat disk-shaped top 57, above the base 20 within said housing 30, outside the patient's body. It extends upwardly above said through-bore 26 located within said housing 30 on said base 20, while said cone shaped lower section 58 remains within the tapered funnel shaped through-bore 26, sealing the lower end of the funnel-shaped through bore 26, wedged below the flange-inwardly projecting flat rim-collar-rib.

In a second embodiment of the inventive Infusion Port 10, where said through-bore 26 is cylindrically shaped, see FIGS. 41-42, the base 20 and the medication delivery apparatus 50 of FIGS. 41 and 42, with the exception of the soft cannula 52, are molded as one unitary piece. Referring more particularly to FIG. 42, the outer lower perimeter of the through-bore 26 adjacent to the bottom surface 20B of said base 20, is provided with a groove 27 and a bushing 28. The soft cannula 52 is pressure fitted within said groove 27, supported by and held in place by the walls of said groove 27 and/or said bushing 28. By comparison, said hard needle 56 is unitarily molded with and extending upwardly from the floor within said housing 30, above and continuous with said through-bore 26.

When the infusion port 10 is not in use, the lid 40 is closed via the hinged joint 42. Its O-Ring 44 fits within the opening of the circular inner cavity 34 within the housing 30 to seal and protect the upwardly protruding hard needle 56 from moisture, dirt and bacteria. When the infusion port 10 is in use, the user lifts the lid 40 by the lip 46 to expose the hard needle 56 within the housing 30. The housing 30 and its hard needle 56 now function as a needleless, medication-pen-receiving socket. The user aligns and slightly pushes down the medication pen within the housing cavity 34 such that the hard needle 56 pierces and penetrates the medication pen's septum or membrane. Upon penetration and depression of the medication pen plunger, the medication pen permits medication to flow through the medication channel formed by the inner bore of said hard needle 56, the through-bore 26, and the soft cannula 52, and into the user. If the housing chamber 34 is provided with an O-ring 44, as discussed in connection with one of the preferred embodiments above, then such O-ring 44 can also function to stabilize the inserted medication pen, prevent its wobbling within the chamber 34 of said housing 30 and provide a more effective delivery of medication. When finished, the user removes the insulin pen from the opening of the inner cavity 34 of the housing 30, leaving behind the hard needle 56 and closing the lid 40 once again. The user has no insulin pen needles to dispose of, does not pollute the environment, and most importantly avoids the daily piercing of their skin, multiple times a day.

An insertion or deployment apparatus 60 is necessary for the final assembly and deployment on the patient, of the inventive infusion port 10. The insertion apparatus 60 comprises an inserter shell 62, a plunger 64 mounted on a stabilizer plate 65, an insertion needle 66 and an inserter cover 68.

The inserter shell 62 is adapted to fit over the housing 30. It has at least having at least two male bosses or protrusions or nubs 76 that correspond to the female indentations 36 on the outer walls of said housing 30. Supported by the inserter shell 62 is a plunger 64 equipped with an insertion needle 66 threaded through the insulin delivery apparatus 50. In the case of the embodiment referenced in FIGS. 5 and 13, the insertion needle 66 is threaded though the hard needle 56, the means to connect the Soft Cannula to the Hard Needle 54, the flat disk shaped top 57, the cone shaped lower end 58 and the soft cannula 52. It is mounted on a stabilizer plate 65 that aligns the medication delivery apparatus 50 in close proximity with the funnel-shaped though-bore 26 within the housing 30 above the base 20.

The plunger 64 comprises a plunger head 64A attached to a plunger shaft 64B. It has the length necessary for the insertion of the medication delivery apparatus 50 into the base 20 of the infusion port 10 and the soft cannula 52 within the patient. The plunger 64 pushes the insertion needle 66 into the patient to deposit the cannula 52 beneath the skin, the hard needle 58 within the housing 30, and the means for connecting both within the base 20, within the funnel-shaped through bore 26. The plunger 64, is located at the center of the inserter shell 62 and inserter cover 68, with the plunger head 64A above the inserter cover, and the rest housed inside the inserter shell 62, supported by the inner stabilizer plate 65.

As was set forth above, the housing 30, is fit within the inserter shell 62. The nubs 76 of the inserter shell are locked into the female indentations 36 of the housing 30. The insertion needle 66 is threaded through the medication delivery apparatus 50 and suspended from the end of the plunger 64 opposite the plunger head 64A, within the inserter shell 62. Once the base 20 is adhered flush to the skin, the insertion apparatus 60 can be deployed. The user pushes down on the plunger head 64A until it meets the inserter cover 68. The plunger 64 has the necessary length to properly insert the cannula 52 beneath the skin. The plunger shaft 64B travels down through the plunger aperture 68A of the inserter cover 68, forcing the insertion needle into the user. The stabilizer plate 65 aligns the insertion needle 66 with the inlet of the through bore 26 as it pierces the skin, to deposit the soft cannula within the user.

The same pressure forces the cone 58 of the medication delivery apparatus 50 into the funnel-shaped through bore. The base 20 is sandwiched between the cone 58 inside the funnel shaped through bore and the disk 57 above the base 20. This locks the medication delivery apparatus 50 into place within the housing. Once inserted, the user pulls up on cap/brim of the inserter cover 68 to pull the plunger out. The plunger 64 pulls the insertion needle 66 with it and leaves the soft cannula within the user, as well as leaving the medication delivery apparatus 50 locked in place with the housing. When pulled up by the user, the inserter cover 68 locks with the inserter shell 62 by the removal nubs of the inserter shell sliding into the female removal indentations located on the fitting of the inserter cover. This is to prevent exposure of the insertion needle. The user then squeezes the sides of the inserter shell to disengage the initial nubs on the inserter shell from the initial indents in the housing. Once the initial nubs are disconnected, the insertion apparatus can be removed and the device is ready to be used. The user is left with the hard needle accessible within the center hole of the housing.

Referring to FIGS. 41 and 42, the second embodiment of the medication delivery apparatus 50, the deployment of the inventive Infusion Port 10 comprises the following steps: removing the protective shield 20C from the bottom surface of the base 20 to expose the adhesive surface 20B; holding the deployment assembly apparatus 60 from the lower wings 74, and breaking off the safety tab 70; placing the adhesive surface 20B of the base 20 within the deployment assembly apparatus 60 on the desired location on the body to access subcutaneous tissue; sliding the fingers upwardly from said lower wings 74 to said upper wings 72, squeezing together said upper wings 72 to pull the nubs out of their corresponding recesses on said housing 30 which concomitantly pressing the plunger down until it comes in contact with the inserter shell to deposit the soft cannula 52 beneath the skin in the subcutaneous region; and de-threading and removing the insertion needle from the deployed medication delivery apparatus by either pulling up on the plunger, or effectively locking the lid in place and removing the entire apparatus away from the body, or pulling the entire apparatus away then locking the lid in place by pulling up on the plunger.

While particular embodiments of the invention are illustrated and descriptions of details provided herein, they are included by way of illustration only and shall not be construed to limit the invention. Since certain revisions may be made with deviations from the scope of the present invention, it is the intent of all matter contained in the above description, or as depicted in the accompanying drawings be interpreted as illustrative and not in the literal sense. Practitioners of the art will realize the sequence of steps and embodiments as depicted in the figures can be revised without deviating from the intent of the present invention and the illustrations contained herein are singular examples of a multitude of possible depictions of the present invention.

What I claim is:

1. An infusion port system for use with a needleless medication pen for the subcutaneous delivery of medicine to a patient, said infusion port system comprising:
a. An infusion port and an insertion and deployment apparatus mounted on and enclosing said infusion port;
b. Said infusion port comprising:
i. a base with a top surface, an adhesive bearing bottom surface, and a removable protective shield covering said adhesive bearing bottom surface;
ii. a through-bore having a bottom open exit port and a top open entry port, said through bore vertically disposed within said base such that said bottom open exit port is located on said adhesive bearing bottom surface and said top open entry port is located on said top surface of said base;
iii. a groove and a bushing located on the outer perimeter of the bottom open exit port of said through-bore;
iv. a housing mounted on said top surface of said base encircling said top open entry port of said through bore and having at least two female indentations located at the exterior surface of the housing;
v. a lid hingedly connected to said housing, said lid and said housing forming a sealable chamber enclosing said top open entry port of said through bore within said housing, when lid is in the closed position on said housing;
vi. a hard needle positioned perpendicularly to the top surface of the base and coaxially aligned with said top open entry port of said through-bore within said housing;
vii. a soft cannula pressure fit within said groove on said bushing, said soft cannula, said through-bore and said hard needle contiguously aligned along a longitudinal axis to form a medication delivery channel; and
c. Said insertion and deployment apparatus having male nubs insertable in the female indentations located on said housing of said infusion port, a safety tab, lower wings, upper wings, a plunger suspended within said insertion and deployment apparatus and capable of being actuated from a first position to a second position, and an insertion needle suspended from said plunger and threaded through said hard needle, said through-bore and said soft cannula;
wherein when the safety tab is broken by holding the lower wings, and the upper wings are squeezed, the plunger can be actuated from a first position to a second position to push the threading needle into the skin and deposit the soft cannula under the skin and wherein said housing acts as a socket for receiving the needleless medicine containing pen which upon its placement on and piercing of its diaphragm by said housing needle within said housing and the pressing of the pen's plunger, medicine flows through the hard needle, the through-bore and the cannula into the patient's body.

2. An infusion port comprising:
a. a base having a bottom surface, a top surface, and a through-bore whose outer perimeter comprises a cannula receiving groove;
b. a housing, mounted on the top surface of the base having a housing chamber sealed with a lid;
c. a housing needle mounted perpendicularly to the top surface of the base within said housing contiguously collinear with said through-bore; and
d. a cannula pressure fitted within the cannula receiving groove and extending downward from the bottom surface of the base, such that the cannula, the through-bore, and the housing needle are co-axially aligned to form a medicine delivery channel;
wherein said housing acts as a socket for receiving a needleless medicine containing pen which upon its placement on and piercing of its diaphragm by said housing needle within said housing and the pressing of the pen's plunger, medicine flows through the medicine channel and out the cannula into the patient's body.
